(19) United States
(12) Patent Application Publication
(10) Pub. No.: US 2023/0175063 A1
(43) Pub. Date: Jun. 8, 2023

(54) EXOSOME MICRORNA AS SCHIZOPHRENIA MARKER AND USE THEREOF

(71) Applicant: Minzu University of China, Beijing (CN)

(72) Inventors: Yong Cheng, Beijing (CN); Yang Du, Beijing (CN); Yang Hu, Beijing (CN); Zongze Cao, Beijing (CN); Yun Yu, Beijing (CN); Xiaowan Li, Beijing (CN); Zexu Wei, Beijing (CN)

(21) Appl. No.: 17/609,294

(22) PCT Filed: May 7, 2020
(86) PCT No.: PCT/CN2020/089019

§ 371 Date: Apr. 11, 2022

(30) Foreign Application Priority Data
May 7, 2019 (CN) 201910376061.0

(51) Int. Cl. C12Q 1/6883

(52) U.S. Cl. CPC C12Q 1/6883; C12Q 2600/158; C12Q 2600/178

(57) ABSTRACT

The present invention provides exosomal microRNAs as a marker for schizophrenia and the use thereof. Specifically, the present invention provides the use of reagent materials and/or instrumentation for detecting microRNA level in a sample from an individual to be tested in the manufacture of a detection system for diagnosing the risk of developing schizophrenia, the microRNA comprising: hsa-miR-206, hsa-miR-145-5p, hsa-miR-133a-3p, hsa miR-143-3p, hsa-miR-144-5p, hsa-miR-144-3p, hsa-miR-184, hsa-miR-499a-5p, hsa-miR-3614-5p, hsa-miR-941, hsa-miR-30c-5p, hsa-miR-339-5p, hsa-miR-30b-5p, and/or hsa-miR-6515-5p. The exosomal miRNAs of the present invention can be used as a biomarker for diagnosing patients with first-episode schizophrenia.

### TECHNICAL FIELD

The present invention relates to a biomarker for diagnosing schizophrenic patients and related applications, more specifically, to the use of exosomal microRNA as a technique for diagnosing suspected schizophrenic patients, and belongs to the technical fields of biology and medicine.

### BACKGROUND

Schizophrenia is a psychiatric disorder characterized by abnormal social behaviors and is a complex mental illness of unknown etiology. Clinical manifestations include disorders in perception, thinking, emotion, and behavior, as well as uncoordinated mental activities, reduced social participation, lack of motivation and the like. People with schizophrenia also tend to have additional mental health problems such as anxiety and depressive states. Symptoms usually emerge gradually from adolescence and continue to develop. Epidemiological surveys show a lifetime prevalence of about 1% in worldwide adult population. It is estimated that there are currently 7-8 million people with schizophrenia in China, and the resulting annual expenditure on medical care and loss of productivity for the patients themselves and their families are staggering. With current treatments only about 20% of people show significant improvement and a few recover completely. Schizophrenia can cause social problems, such as long-term unemployment, poverty and homelessness. The average life expectancy of people with schizophrenia is ten to twenty-five years shorter than that of the general population. Another result is that suicide rate is higher in schizophrenics than in the normal population (about 5%). In 2015, approximately 17,000 people worldwide died from behaviors related to or caused by schizophrenia.

Currently, the clinical diagnosis of schizophrenia is mainly based on the patient's detailed medical history and psychiatric symptoms, and a comprehensive judgment is made by subjective scores such as the PANSS scale and ICD10. Due to the different subjective experiences of physicians, the diagnostic results also vary.

In order to make the diagnosis of schizophrenia more objective, reduce human factors, and improve the consistency and accuracy of diagnosis, scientific researchers around the world have been working in recent years to find biomarkers of schizophrenia and establish effective detection methods.

### SUMMARY OF THE INVENTION

An object of the present invention is to provide new biomarkers for schizophrenia.

Another object of the present invention is to provide the related use of the biomarkers for schizophrenia.

In an investigation, the inventors of the present invention extracted exosomes and total RNA from peripheral blood of subjects, subjected the samples to whole microRNA sequencing analysis, and statistically analyzed the expression abundance. In the analysis, three miRNAs (hsa-miR-206, hsa-miR-145-5p, hsa-miR-133a-3p) that were significantly differentially expressed between healthy individuals and schizophrenia patients were selected for expression in the first batch of 46 samples (training set). Seven significantly different miRNAs (hsa-miR-206, hsa-miR-145-5p, hsa-miR-133a-3p, hsa-miR-142-3p, hsa-miR-619-5p, hsa-miR-144-3p, has-miR-483-5p) were selected from 49 samples in the second batch of data (test set). After combining the data from the two batches, six significantly different miRNAs (hsa-miR-206, hsa-miR-145-5p, hsa-miR-133a-3p, hsa-miR-144-5p, hsa-miR-144-3p, hsa-miR-184) were finally selected. During further studies, the first batch of data (training set) was trained with a random forest algorithm model to select miRNA markers (hsa-miR-206, hsa-miR-133a-3p, hsa-miR-143-3p, hsa-miR-144-5p, hsa-miR-499a-5p, hsa-miR-3614-5p, hsa-miR-941, hsa-miR-30c-5p, hsa-miR-339-5p, hsa-miR-30b-5p, hsa-miR-6515-5p) to build a classifier with an AUC index of up to 94.14% and a sensitivity and specificity of 78.3% and 95.7%, respectively. The ROC curves obtained by building the classifier on the second batch of data (test set) had a sensitivity and specificity of 76.9% and 78.3%, respectively.

Thus, in one aspect, the present invention provides the use of microRNAs selected from the group consisting of:

hsa-miR-206;

hsa-miR-145-5p;

hsa-miR-133a-3p;

hsa-miR-143-3p;

hsa-miR-144-5p;

hsa-miR-144-3p;

hsa-miR-184;

hsa-miR-499a-5p;

hsa-miR-3614-5p;

hsa-miR-941;

hsa-miR-30c-5p;

hsa-miR-339-5p;

hsa-miR-30b-5p; and/or

hsa-miR-6515-5p,

as a biomarker for diagnosing schizophrenia.

In another aspect, the present invention also provides the use of reagent materials and/or instrumentation for detecting the microRNA level in a sample from an individual to be tested in the manufacture of a detection system for diagnosing the risk of developing schizophrenia, the microRNA comprising:

hsa-miR-206;

hsa-miR-145-5p;

hsa-miR-133a-3p;

hsa-miR-143-3p;

hsa-miR-144-5p;

hsa-miR-144-3p;

hsa-miR-184;

hsa-miR-499a-5p;

hsa-miR-3614-5p;

hsa-miR-941;

hsa-miR-30c-5p;

hsa-miR-339-5p;

hsa-miR-30b-5p; and/or

hsa-miR-6515-5p.

According to a specific embodiment of the present invention, the microRNA level in the present invention includes a peripheral blood expression level and, in a more specific embodiment, is a peripheral blood serum expression level.

According to a specific embodiment of the present invention, the microRNA detected in the present invention comprises one or more selected from the group consisting of hsa-miR-206, hsa-miR-145-5p, hsa-miR-133a-3p, hsa-miR-144-5p, hsa-miR-144-3p, and hsa-miR-184. Preferably, the microRNA comprises one or more selected from the group consisting of hsa-miR-206, hsa-miR-145-5p, hsa-miR-133a-3p. Further, the microRNA detected further comprise one or more of hsa-miR-143-3p, hsa-miR-499a-5p, hsa-miR-3614-5p, hsa-miR-941, hsa-miR-30c-5p, hsa-miR-339-5p, hsa-miR-30b-5p, and hsa-miR-6515-5p. Further, the microRNA detected may further comprise one or more selected from the group consisting of hsa-miR-142-3p, hsa-miR-619-5p, hsa-miR-483-5p.

According to a specific embodiment of the present invention, in the present invention, an expression level of hsa-miR-206, hsa-miR-145-5p, hsa-miR-133a-3p, hsa-miR-619-5p and/or hsa-miR-184 is elevated, and the individual to be tested has an increased risk of developing schizophrenia.

According to a specific embodiment of the present invention, in the present invention, an expression levels of hsa-miR-142-3p, hsa-miR-144-3p, hsa-miR-483-5p and/or hsa-miR-144-5p is reduced, and the individual to be tested has an increased risk of developing schizophrenia.

In another aspect, the present invention also provides a detection system for assessing risk of developing schizophrenia, which comprises: reagent materials and/or instrumentation for detecting the level of microRNA selected from the group consisting of:

hsa-miR-206;

hsa-miR-145-5p;

hsa-miR-133a-3p;

hsa-miR-143-3p;

hsa-miR-144-5p;

hsa-miR-144-3p;

hsa-miR-184;

hsa-miR-499a-5p;

hsa-miR-3614-5p;

hsa-miR-941;

hsa-miR-30c-5p;

hsa-miR-339-5p;

hsa-miR-30b-5p; and/or

hsa-miR-6515-5p.

According to a specific embodiment of the present invention, the detection system for assessing risk of developing schizophrenia of the present invention comprises:

a detection unit comprising reagent materials and/or instrumentation for detecting the microRNA level;

an analysis unit for analyzing test results of the detection unit and assessing the risk of schizophrenia in the individual to be tested.

According to a specific embodiment of the present invention, for the detection system for assessing risk of developing schizophrenia of the present invention, the sample is peripheral blood.

According to a specific embodiment of the present invention, for the detection system for assessing risk of developing schizophrenia of the present invention, the schizophrenia is first-episode schizophrenia.

According to a specific embodiment of the present invention, for the detection system for assessing risk of developing schizophrenia of the present invention, an expression level of hsa-miR-206, hsa-miR-145-5p, hsa-miR-133a-3p, hsa-miR-619-5p and/or hsa-miR-184 is elevated, and the individual to be tested has an increased risk of developing schizophrenia.

According to a specific embodiment of the present invention, for the detection system for assessing risk of developing schizophrenia of the present invention, an expression levels of hsa-miR-142-3p, hsa-miR-144-3p, hsa-miR-483-5p and/or hsa-miR-144-5p is reduced, and the individual to be tested has an increased risk of developing schizophrenia.

The microRNA expression level can be detected by any available techniques in the art.

According to a specific embodiment of the present invention, the reagent materials and/or instrumentation for detecting the expression level of microRNA in the present invention may be any reagent materials and/or instrumentation used in any available techniques for detecting the expression level of microRNAs.

The detection system for assessing risk of developing schizophrenia of the present invention can be a virtual device, as long as it can perform the function of the detection unit as well as the assessment unit. The detection unit may include a variety of detection reagent materials and/or detection instruments and equipments and the like; the data analysis unit may be any computing devices, modules or virtual devices that allow the analysis and processing of the test results of the detection unit to derive a schizophrenia risk assessment status. For example, a corresponding data chart may be preliminarily set up from various possible test results and corresponding risk profiles, and the results of the detection unit can be compared to the data chart to obtain a risk assessment for schizophrenia.

The application of the techniques of the present invention can provide positive assistance in the pathogenesis of schizophrenia and in the investigation of novel targeted therapies.

### BRIEF DESCRIPTION OF THE DRAWINGS

FIG. 1 shows a photograph observed by transmission electron microscopy of the extracted exosomes in Example 1 of the present invention.

FIG. 2 shows a plot of the particle size distribution of the extracted exosomes from Example 1 of the present invention detected by Zetaview.

FIG. 3A shows the MA-plot of differential analysis of the first batch of data in Example 2 of the present invention. FIG. 3B shows the MA-plot of differential analysis of the second batch of data in the Example of the present invention. FIG. 3C shows the MA-plot of the differential analysis of the combined two batches of data in the Example of the present invention. In the figures, the X-axis is the normalized read count value, and the Y-axis is the logarithmic value of the difference multiplier between the first-episode schizophrenia patient group and the healthy control group, and a value of less than 0 means down-regulation and a value of greater than 0 means up-regulation. Each dot in the plot represents a miRNA, and the red dots are significantly different miRNAs.

FIG. 4A shows the ROC curve of the classification results of the training set in Example 3 of the present invention, with an AUC index of about 94.14% and the green area in the curve showing the 95% confidence interval. FIG. 4B shows a box plot of the predicted probability of the training set samples belonging to the schizophrenia group. FIG. 4C shows a scatter plot of the predicted probability of the training set samples belonging to the schizophrenia group, with the x-axis sorted from the lowest to the highest.

FIG. 5A shows the ROC plot of classification results of the test set with an AUC index of approximately 75.33%. FIG. 5B shows a box plot of the predicted probability of the test set samples belonging to the schizophrenia group. FIG. 5C shows a scatter plot of the predicted probability of the test set samples belonging to the schizophrenia group, with the x-axis sorted from the lowest to the highest.

### DETAILED DESCRIPTION OF THE INVENTION

The implementation of the technical solutions of the present invention and the beneficial effects thereof are hereinafter described in details by means of specific examples. However, these cannot be construed as limitation in any way to the implementable scope of the present invention.

### EXAMPLE 1

Subjects were 49 patients with first-episode unmedicated schizophrenia admitted to the Foshan Third People's Hospital, and 46 age- and sex-matched healthy volunteers were recruited as control. All physicians involved in the diagnostic work were qualified as psychiatrists and had more than 10 years of experience in psychiatric practice, were skilled in the use of the SCID-1 checklist, and proficient in the ICD-10 and DSM-V diagnostic criteria. They assessed the psychopathological status of patients by using the Positive and Negative Symptom Scale (PANSS), while operating in a uniform manner and meeting the requirements for consistency testing (kappa=0.68-0.82). Schizophrenic patients with comorbidities were excluded. All participants gave written informed consent prior to inclusion in the study. The study protocol was approved by the Ethics Committee of Foshan Third People's Hospital.

1. Exosome Extraction

Approximately 4 ml of peripheral blood was collected from each subject and allowed to clot at room temperature for 1 hour. The samples were then centrifuged at 3000×g for 10 minutes to obtain serum. The sera were then stored in a low temperature refrigerator at −80° C. or analyzed directly.

The filtration was performed using a 0.22 μm membrane, and the filtrate was centrifuged at 10,000 G for 1 hour. The exosome suspension was washed by concentrated ultrafiltration using a 30 kd ultrafiltration tube, and 20 ml PBS was added before centrifugation was carried out at 110,000 g for 70 minutes for the first time. The supernatant was removed, and 5 mL of solution was remained at the bottom, and was then resuspended and gently blown before centrifugation was carried out at 110,000 g for 70 minutes for the second time. After centrifugation, a pale yellow pellet was visible at the bottom of the tube, the supernatant was removed, and 1 mL of solution was remained at the bottom and was then resuspended with PBS while replenishing to the specified volume of the centrifuge tube. Centrifugation was carried out at 110,000 g for 70 minutes for the third time. The supernatant was carefully removed, and the collected exosomes were transferred to a 1.5 mL Eppendorf tube and stored at −80° C. in the refrigerator.

2. Identification of Exosomes

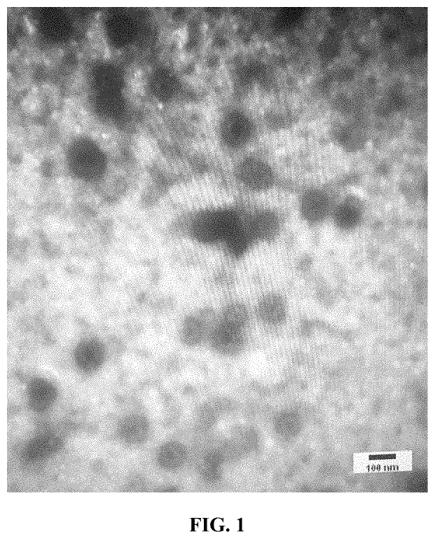

2.1 Morphological analysis of exosomes (electron microscopic verification): the extracted exosomes were resuspended with PBS, and 20 μL of the suspension was dropped onto a copper mesh with a pore size of 2 nm, and left to stand for 2 minutes at room temperature. The liquid was blotted out from the side of the mesh with filter paper, and negative staining with 30 μL of a 3% phosphotungstic acid solution was conducted for 5 minutes at room temperature. The negative staining solution was blotted out with filter paper, dried at room temperature, and photographed by transmission electron microscopy. The results are shown in FIG. 1, indicating that the vesicle sizes and structures are consistent with the typical morphology of exosomes.

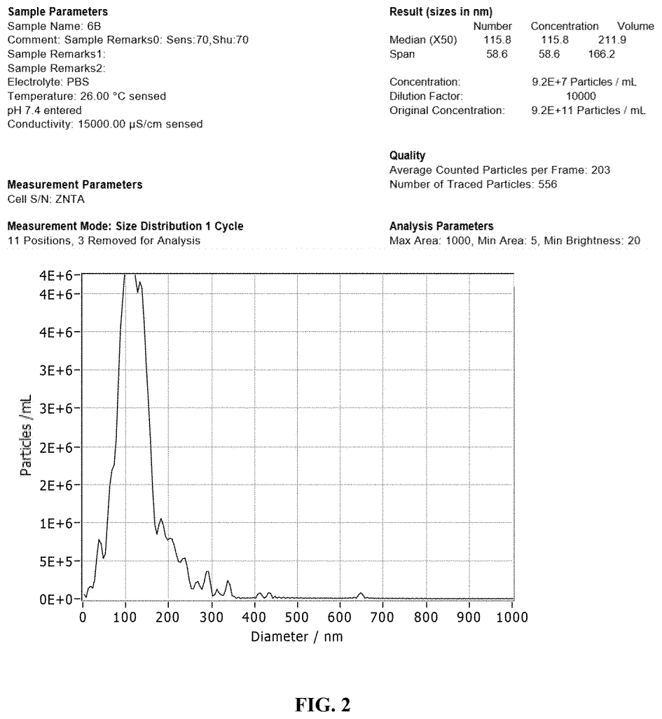

2.2 Zetaview detection of exosome particle size: the collected exosomes were diluted with PBS to a particle concentration of 10⁶/mL, injected with a 1 mL syringe into a nanoparticle tracking analyzer for analysis, and the analysis data were saved. The analyzer operating parameters and results are shown in FIG. 2, indicating that the particle sizes are in accordance with typical exosome characteristics, with a concentration of 9.2E+11/mL.

### EXAMPLE 2

After the exosome extraction and total RNA extraction of samples in Example 1, the samples were analyzed by whole microRNA sequencing and the expression abundance was statistically analyzed, and the data were expressed as mean±standard deviation. Statistical analysis was performed using a two-tailed t-test, and p<0.05 was considered statistically significant.

1. First Batch Screening for Significantly Different miRNAs

In this example, the first batch of data (training set) had 46 samples of expression, with 23 healthy controls and 23 first-episode schizophrenic patients. In this example, low expression miRNAs with a mean value of TPM <10 in all samples were filtered out, and a total of 353 miRNAs were entered into the differential analysis.

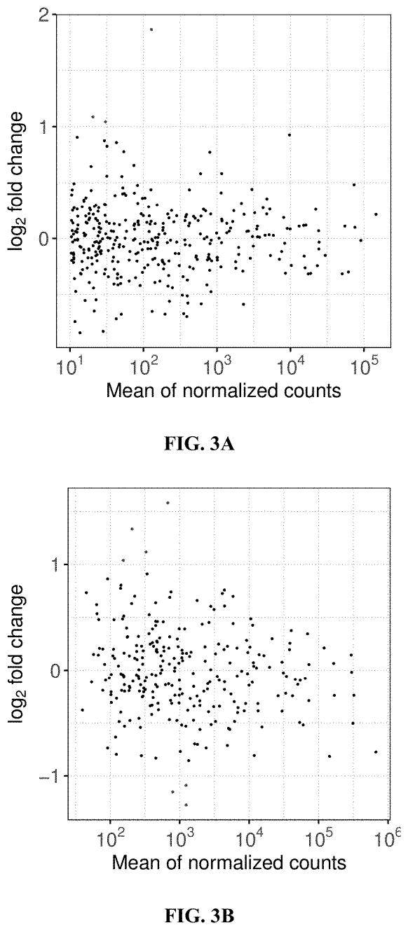

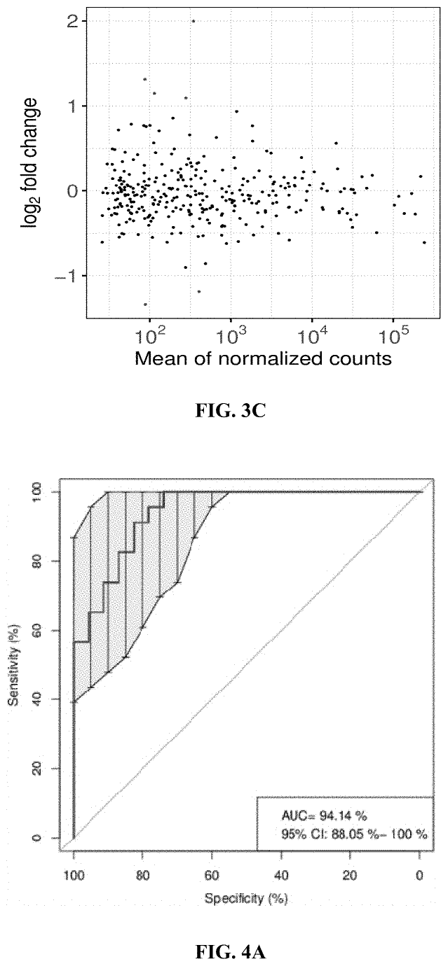

In this example, DESeq2 was used for the difference analysis, and miRNAs with a Q value of Qvalue (adjust-p) ≤0.05 and a difference multiplier ≥2 were defined as significantly different miRNAs, and three significantly different miRNAs were finally selected (Table 1), with the MA-plot thereof shown in FIG. 3A.

TABLE 1
Significantly different miRNAs from the first batch of data

| | Sequencing | Sequencing | | | | |
|---|---|---|---|---|---|---|
| | expression | expression | | | | |
| | levels in | levels in | | | | |
| | disease | control | Difference | | | |
| miRNA | group | group | multiplier | P value | Q value | Status |
| hsa-miR-206 | 391.1 | 35.0 | 1.865226 | 1.36e−11 | 4.79e−09 | High |
| | | | | | | expression |
| hsa-miR-145-5p | 58.1 | 21.2 | 1.043607 | 1.54e−05 | 0.001816 | High |
| | | | | | | expression |
| hsa-miR-133a-3p | 38.0 | 15.3 | 1.086098 | 8.92e−06 | 0.001574 | High |
| | | | | | | expression |

In FIG. 3A, the X-axis indicates the normalized read count value, and the Y-axis indicates the logarithmic value of the difference multiplier between the first-episode schizophrenia patient group and the healthy control group. A value of less than 0 means down-regulation, and a value of greater than 0 means up-regulation. Each dot in the graph represents a miRNA, and the red dots are significantly different miRNAs.

2. Second Batch Validation of Significantly Different miRNAs

The second data set (test set) had 49 samples, with 23 healthy controls and 23 first-episode schizophrenic patients. By using the same method as the first batch of data, seven significantly different miRNAs were finally selected from the second batch of data (Table 2), with the MA-plot thereof shown in FIG. 3B.

The differential miRNAs identified in the first batch of data (hsa-miR-206, hsa-miR-145-5p, hsa-miR-133a-3p) were all found in the second batch of data. Other differential miRNAs screened in the second batch of data (hsa-miR-142-3p, hsa-miR-619-5p, hsa-miR-144-3p, hsa-miR-483-5p) were also expressed in the first batch of data, but the differences were not significant.

TABLE 2
Significantly different miRNAs from the second batch data

| | Sequencing | Sequencing | | | | |
|---|---|---|---|---|---|---|
| | expression | expression | | | | |
| | levels in | levels in | | | | |
| | disease | control | Difference | | | |
| miRNA | group | group | multiplier | P value | Q value | Status |
| hsa-miR-145-5p | 352.4 | 136.2 | 1.335239 | 1.80E−07 | 2.39E−05 | High |
| | | | | | | expression |
| hsa-miR-206 | 1498.8 | 259.0 | 1.581903 | 4.37E−06 | 0.00029 | High |
| | | | | | | expression |
| hsa-miR-142-3p | 1306.3 | 2866.6 | −1.08891 | 1.13E−05 | 0.000497 | Low |
| | | | | | | expression |
| hsa-miR-619-5p | 184.4 | 93.3 | 1.038452 | 0.000148 | 0.004012 | High |
| | | | | | | expression |
| hsa-miR-144-3p | 1122.8 | 3091.4 | −1.27548 | 0.000151 | 0.004012 | Low |
| | | | | | | expression |
| hsa-miR-133a-3p | 625.7 | 254.6 | 1.117833 | 0.0003 | 0.006686 | High |
| | | | | | | expression |
| hsa-miR-483-5p | 32665.6 | 67904.8 | −1.150913 | 0.00048 | 0.009788 | Low |
| | | | | | | expression |

3. Identification of Differential miRNAs by Combining the Two Batches of Data

In this example, the two batches of data were further combined (i.e., 46 healthy controls and 49 first-episode schizophrenic patients) and the same method as that for the first batch of data was used for difference analysis, and 6 significantly different miRNAs were finally selected (Table 3), with the MA-plot thereof shown in FIG. 3C. The three differential miRNAs (hsa-miR-206, hsa-miR-145-5p, hsa-miR-133a-3p) identified in the first batch of data were also found in the combined data.

TABLE 3
Significantly different miRNAs after combination of data from two batches

| | Sequencing | Sequencing | | | | |
|---|---|---|---|---|---|---|
| | expression | expression | | | | |
| | levels in | levels in | | | | |
| | disease | control | Difference | | | |
| miRNA | group | group | multiplier | P value | Q value | Status |
| hsa-miR-206 | 978.8 | 147.0 | 1.997652 | 5.79E−15 | 1.83E−12 | High |
| | | | | | | expression |
| hsa-miR-144-5p | 81.6 | 284.6 | −1.33957 | 4.60E−12 | 7.25E−10 | Low |
| | | | | | | expression |
| hsa-miR-145-5p | 214.3 | 78.7 | 1.313401 | 3.95E−11 | 4.14E−09 | High |
| | | | | | | expression |
| hsa-miR-133a-3p | 349.8 | 135.0 | 1.148248 | 2.54E−06 | 0.000134 | High |
| | | | | | | expression |
| hsa-miR-144-3p | 615.3 | 1579.8 | −1.18817 | 4.16E−06 | 0.000187 | Low |
| | | | | | | expression |
| hsa-miR-184 | 550 | 433.9 | 1.093833 | 1.33E−05 | 0.000382 | High |
| | | | | | | expression |

In this example, the three significantly different miRNAs (hsa-miR-206, hsa-miR-145-5p, hsa-miR-133a-3p) identified in the first batch of data were found in the second batch of data and the combined data of the two batches, indicating that these three miRNAs were true positives.

### EXAMPLE 3

The first batch of data (training set) of the project, with 23 schizophrenia samples and 23 control samples, were trained by using the random forest algorithm model to screen out miRNA markers, and these markers were used as classifiers for the second batch of data (test set) to verify whether their classification was valid.

Result analysis:

1. Screening of miRNA Markers

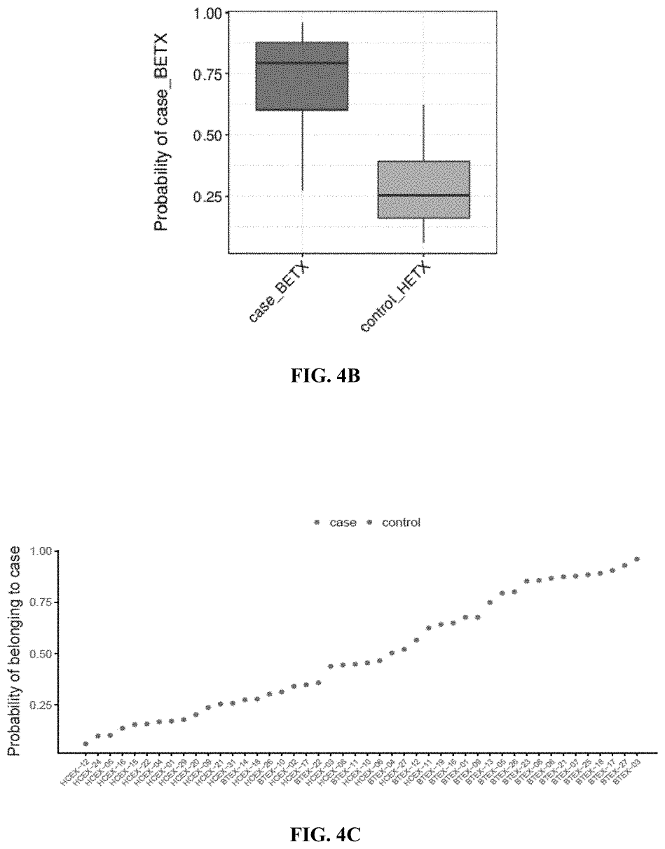

From the original miRNAs in the training set, the miRNAs with low expression were filtered (mean TPM <10 in all samples) before classifying the remaining miRNAs by using the random forest algorithm, and the classifiers were indicated. Table 4 shows the 11 miRNA markers selected with the classifiers and the expression levels thereof (for positive difference multiples, the higher the expression level, the higher the risk of developing the disease; for negative difference multiples, the higher the expression level, the lower the risk of developing the disease). Their ROC curves and the distribution of the probability of being determined as schizophrenic for each sample are shown in FIGS. 4A-4C. The true and false positive rates and true and false negative rates were also counted, with a cutoff of a probability of 0.519337017.

TABLE 4
miRNA markers selected from the training set

| | Mean expression | Mean expression | | |
|---|---|---|---|---|
| | level in disease | level in healthy | Difference | |
| miRNA | group | control group | multiplier | P value |
| hsa-miR-206 | 294.8764 | 29.6888 | 3.312119676 | 1.51E−11 |
| hsa-miR-133a-3p | 29.1805 | 10.9612 | 1.412598855 | 5.31E−05 |
| hsa-miR-143-3p | 13049.239 | 5812.8824 | 1.166640048 | 0.000188555 |
| hsa-miR-144-5p | 17.2568 | 36.0789 | −1.063990389 | 0.000575185 |
| hsa-miR-499a-5p | 16.3996 | 7.13 | 1.201686645 | 0.000705199 |
| hsa-miR-3614-5p | 92.049 | 50.7929 | 0.857775202 | 0.001564995 |
| hsa-miR-941 | 1386.9193 | 830.313 | 0.740156653 | 0.001984757 |
| hsa-miR-30c-5p | 263.1816 | 464.6396 | −0.820053488 | 0.002159794 |
| hsa-miR-339-5p | 65.5626 | 84.175 | −0.360518748 | 0.019090136 |
| hsa-miR-30b-5p | 8.6858 | 13.9844 | −0.687087718 | 0.033913143 |
| hsa-miR-6515-5p | 9.3712 | 12.5392 | −0.420139603 | 0.067131331 |

True positive rate (sensitivity): 18/23=78.3%;

True negative rate (specificity): 22/23=95.7%;

False positive rate (misdiagnosis rate): 1/23=4.3%;

False negative rate (missed-diagnosis rate): 5/23=21.7%.

2. Validation of miRNA Markers

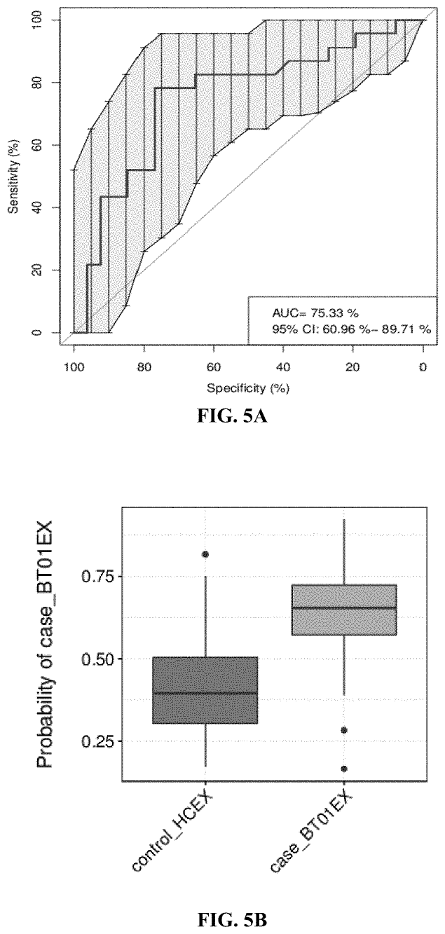

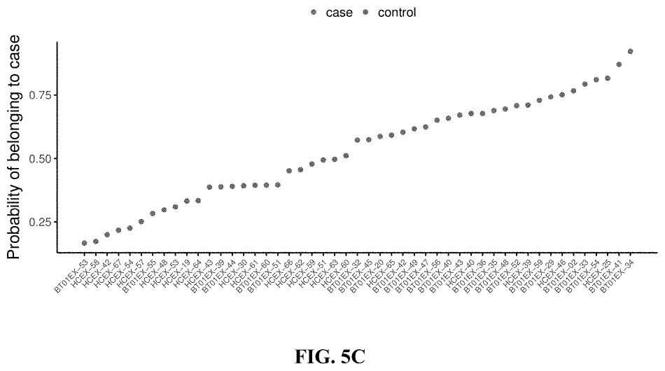

For the test set (26 schizophrenia cases and 23 normal controls), the low-expression miRNAs were filtered (268 miRNAs remaining) by the same method, and then the 11 miRNA markers selected from the training set were used for classification of the samples. The results are shown in FIG. 5A-5C. The true and false positive rates and true and false negative rates of the test set were also counted, with the cutoff set in the training set of a probability of 0.519337017.

True positive rate (sensitivity): 20/26=76.9%;

True negative rate (specificity): 18/23=78.3%;

False positive rate (misdiagnosis rate): 5/26=21.7%;

False negative rate (missed-diagnosis rate): 6/26=23.1%.

Table 5 shows the test set validation results.

TABLE 5
Validation results from the test set

| | | Healthy control | Difference |
|---|---|---|---|
| miRNA | Disease group | group | multiplier |
| hsa-miR-144-5p | 13.06329 | 36.44133 | −1.480057328 |
| hsa-miR-206 | 202.068 | 25.74682 | 2.97237471 |
| hsa-miR-199b-5p | 20.66225 | 11.85472 | 0.801535776 |
| hsa-miR-143-3p | 8370.737 | 4759.465 | 0.814555237 |
| hsa-miR-133a-3p | 39.51876 | 15.15253 | 1.382978981 |
| hsa-miR-144-3p | 50.54968 | 149.6847 | −1.5661529 |

Conclusion:

By using the first batch of data as a training set for miRNA marker screening, an ideal ROC graph may be obtained, with an AUC index of up to 94.14%, and relatively high sensitivity and specificity of 78.3% and 95.7%, respectively, from the statistics thereof. The selected markers were applied to the second training set, and the sensitivity and specificity were 76.9% and 78.3%, respectively.

The above results suggest that the exosomal miRNA screened in the present invention can be used as a biomarker for diagnosing patients with first-episode schizophrenia.

1. A method for diagnosing schizophrenia, comprising using a microRNA selected from the group consisting of:
hsa-miR-206;
hsa-miR-145-5p;
hsa-miR-133a-3p;
hsa-miR-143-3p;
hsa-miR-144-5p;
hsa-miR-144-3p;
hsa-miR-184;
hsa-miR-499a-5p;
hsa-miR-3614-5p;
hsa-miR-941;
hsa-miR-30c-5p;
hsa-miR-339-5p;
hsa-miR-30b-5p; and/or
hsa-miR-6515-5p,
as a biomarker for diagnosing schizophrenia.

2. A method for diagnosing the risk of developing schizophrenia, comprising detecting the microRNA level in a sample from an individual to be tested, wherein the microRNA comprising:
hsa-miR-206;
hsa-miR-145-5p;
hsa-miR-133a-3p;
hsa-miR-143-3p;
hsa-miR-144-5p;
hsa-miR-144-3p;
hsa-miR-184;
hsa-miR-499a-5p;
hsa-miR-3614-5p;
hsa-miR-941;
hsa-miR-30c-5p;
hsa-miR-339-5p;
hsa-miR-30b-5p; and/or
hsa-miR-6515-5p.

3. The method according to claim 2, wherein the microRNA level includes a peripheral blood expression level or serum expression level.

4. The method according to claim 2, wherein the microRNA comprises one or more selected from the group consisting of hsa-miR-206, hsa-miR-145-5p, hsa-miR-133a-3p, hsa-miR-144-5p, hsa-miR-144-3p, and hsa-miR-184; and
wherein, preferably, the microRNA comprises one or more selected from the group consisting of hsa-miR-206, hsa-miR-145-5p, hsa-miR-133a-3p.

5. The method according to claim 4, wherein the microRNA further comprises one or more selected from the group consisting of hsa-miR-143-3p, hsa-miR-499a-5p, hsa-miR-3614-5p, hsa-miR-941, hsa-miR-30c-5p, hsa-miR-339-5p, hsa-miR 30b-5p, hsa-miR-6515-5p; and
wherein, the microRNA may further comprise one or more selected from the group consisting of hsa-miR-142-3p, hsa-miR-619-5p, hsa-miR-483-5p.

6. The method according to claim 2, wherein an expression level of hsa-miR-206, hsa-miR-145-5p, hsa-miR-133a-3p, hsa-miR-619-5p and/or hsa-miR-184 is elevated, and the individual to be tested has an increased risk of developing schizophrenia.

7. The method according to claim 2, wherein an expression levels of hsa-miR-142-3p, hsa-miR-144-3p, hsa-miR-483-5p and/or hsa-miR-144-5p is reduced, and the individual to be tested has an increased risk of developing schizophrenia.

8. A detection system for assessing the risk of developing schizophrenia, which comprises: reagent materials and/or instrumentation for detecting the level of microRNA selected from the group consisting of:
hsa-miR-206;
hsa-miR-145-5p;
hsa-miR-133a-3p;
hsa-miR-143-3p;
hsa-miR-144-5p;
hsa-miR-144-3p;
hsa-miR-184;
hsa-miR-499a-5p;
hsa-miR-3614-5p;
hsa-miR-941;
hsa-miR-30c-5p;
hsa-miR-339-5p;
hsa-miR-30b-5p; and/or
hsa-miR-6515-5p
in a sample from an individual to be tested.

9. The detection system according to claim 8, which comprises:
a detection unit comprising the reagent materials and/or instrumentation for detecting the microRNA level;
an analysis unit for analyzing the test results from the detection unit and assessing the risk of schizophrenia of the individual to be tested.

10. The detection system according to claim 8, wherein the sample is peripheral blood; the schizophrenia is first-episode schizophrenia;
wherein an expression level of hsa-miR-206, hsa-miR-145-5p, hsa-miR-133a-3p, hsa-miR-619-5p and/or hsa-miR-184 is elevated, and the individual to be tested has an increased risk of developing schizophrenia;
an expression levels of hsa-miR-142-3p, hsa-miR-144-3p, hsa-miR-483-5p and/or hsa-miR-144-5p is reduced, and the individual to be tested has an increased risk of developing schizophrenia.
